(19) United States
(12) Patent Application Publication
(10) Pub. No.: US 2026/0347776 A1
(43) Pub. Date: Nov. 5, 2026

(54) Method of Producing Regulatory Dendritic Cells

(71) Applicants: University of Pittsburgh -Of the Commonwealth System of Higher Education, Pittsburgh, PA (US); Ri.MED Foundation, Palermo (IT); Istituto Mediterraneo per i Trapianti e Terapie ad Alta Specializzazione S.r.I., Palermo (IT)

(72) Inventors: Angus W. Thomson, Pittsburgh, PA (US); Ester Badami, Palermo (IT); Rosalia Busà, Palermo (IT); Pier Giulio Conaldi, Palermo (IT); Fadi G. Lakkis, Pittsburgh, PA (US)

(21) Appl. No.: 19/486,420

(22) PCT Filed: May 24, 2024
(86) PCT No.: PCT/US2024/030950

§ 371 Date: Nov. 21, 2025

Related U.S. Application Data
(60) Provisional application No. 63/504,494, filed on May 26, 2023.

(51) Int. Cl. C12N 5/0784; A61K 35/15

(52) U.S. Cl. CPC C12N 5/0639; A61K 35/15; C12N 2500/38; C12N 2501/22; C12N 2501/2304; C12N 2501/231; C12N 2506/115

(57) ABSTRACT

Provided herein are methods of producing regulatory dendritic cells from donor organ perfusate, and use of the donor regulatory dendritic cells for transplantation.

### CROSS-REFERENCE TO RELATED APPLICATION

This application claims priority to U.S. Provisional Patent Application No. 63/504,494 filed May 26, 2023, entitled “Method of Producing Regulatory Dendritic Cells”, the disclosure of which hereby incorporated by reference in its entirety.

A method of producing regulatory dendritic cells (DCregs) is provided. Methods of use of those DCregs, e.g., in transplantation, also are provided.

Current immunosuppressive (IS) regimens utilized to prevent body organ allograft rejection have well-recognized, unwanted effects, that include increased threat of infection and specific types of cancers, metabolic disorders, coronary disease, and renal failure. Early weaning of organ recipients by use of cellular therapy embodied by DCregs is a desirable approach for the minimization or withdrawal of immunosuppressive drugs to establish operational tolerance and prevent graft rejection.

“Regulatory” dendritic cells (DCregs, also referred to as “tolerogenic DCs”) play central roles in maintaining self-tolerance in the healthy steady-state. These regulatory innate immune cells can subvert naïve and memory T-cell responses by various mechanisms. DCregs can induce or restore T-cell tolerance in many animal models of autoimmune disease or organ transplant rejection, including autoimmune hepatitis following transplantation (see, e.g., Harputluoglu M, et al. Autoimmune hepatitis and liver transplantation: Indications, and recurrent and de novo autoimmune hepatitis. World J Transplant. 2022 Mar. 18; 12 (3):59-64 and Liberal R, et al. Recurrence of autoimmune liver disease and inflammatory bowel disease after pediatric liver transplantation. Liver Transpl. 2016 September; 22 (9):1275-83). In experimental transplantation, both allogeneic (donor-derived) DCregs and donor Ag-pulsed or un-pulsed host (autologous) DCregs are effective in rodents. We have shown that high-purity DCregs meeting a range of quality criteria can readily be generated from circulating blood monocytes under Good Manufacturing Practice conditions to meet target cell numbers for infusion into prospective organ transplant recipients (Zahorchak A F, DeRiggi M L, Muzzio J L, Sutherland V, Humar A, Lakkis F G, Hsu Y S, Thomson A W. Manufacturing and validation of Good Manufacturing Practice-compliant regulatory dendritic cells for infusion into organ transplant recipients. Cytotherapy. 2023 April; 25 (4):432-441).

Although generating DCregs from peripheral blood is a valid approach, the largest source of organ tissue is from deceased donors, from which peripheral blood typically cannot be obtained prior to organ removal and transplantation.

### SUMMARY

A method of preparing donor regulatory dendritic cells (donor DCregs) is provided. The method comprising, isolating monocytes (e.g., CD14+ monocytes) from perfusate obtained from perfusion of donor tissue and differentiating the monocytes to a DCreg phenotype.

A transplantation method also is provided. The method comprising: obtaining a donor organ from a donor; perfusing the donor organ with a perfusion solution; isolating monocytes (e.g., CD14+ monocytes) from the perfusion solution; differentiating the monocytes to a DCreg phenotype thereby producing DCregs; implanting the donor organ into a recipient patient; prior to, during, or after implantation of the donor organ into the recipient patient, administering the DCregs to the patient, thereby inducing tolerance to the donor organ in the recipient patient.

The following numbered clauses illustrate various exemplary aspects, embodiment, or examples of the present invention.

Clause 1. A method of preparing donor regulatory dendritic cells (donor DCregs), comprising, isolating monocytes (e.g., CD14+ monocytes) from perfusate obtained from perfusion of donor tissue, and differentiating the monocytes to a DCreg phenotype.

Clause 2. The method of clause 1, wherein the donor graft tissue is an organ.

Clause 3. The method of clause 2, wherein the organ is selected from: liver, kidney, pancreas, heart, lung, intestine, bone, bone marrow, connective tissue, or a vascularized composite graft.

Clause 4. The method of clause 2, wherein the organ is liver.

Clause 5. The method of clause 2, wherein the organ is lung, pancreas, intestine, heart, or kidney.

Clause 6. The method of any one of clauses 1-5, wherein the donor organ is an allograft.

Clause 7. The method of any one of clauses 1-6, wherein the DCregs are prepared by culturing the monocytes in media containing IL4 and GM-CSF.

Clause 8. The method of clause 7, wherein the media further comprises one or more additional immunosuppressive compounds, such as IL-10, Vitamin D3, a combination of IL10 and Vitamin D3, an anti-inflammatory cytokine, or an immunosuppressive or anti-inflammatory drug, such as dexamethasone or rapamycin.

Clause 9. The method of any one of clauses 1-8, wherein the monocytes are obtained by apheresis of the perfusion solution after it passes through the donor organ.

Clause 10. The method of any one of clauses 1-9, wherein the monocytes are obtained by a method comprising elutriation of cells obtained from the perfusion solution.

Clause 11. The method of any one of clauses 1-9, wherein the monocytes are obtained by elutriation of leukocytes obtained by apheresis.

Clause 12. The method of clause 10 or 11, further comprising, after elutriation, affinity-purification of the monocytes by CD14 affinity or depletion of neutrophils by CD15 and/or CD66b affinity, such as by magnetic bead separation.

Clause 13. The method of any one of clauses 1-12, wherein the monocytes are obtained from a back-table perfusate fraction and/or from a container in which the donor organ is transferred from the donor to recipient patient.

Clause 14. The method of any one of clauses 1-13, wherein the monocytes are stored frozen and thawed prior to differentiating to a DCreg phenotype.

Clause 15. The method of any one of clauses 1-13, wherein a first portion of the monocytes are stored frozen and thawed prior to differentiating to a DCreg phenotype and a second portion of the monocytes are differentiating to a DCreg phenotype without freezing.

Clause 16. A transplantation method comprising:
obtaining a donor organ from a donor;
perfusing the donor organ with a perfusion solution;
isolating monocytes (e.g., CD14+ monocytes) from the perfusion solution;
differentiating the monocytes to a DCreg phenotype thereby producing DCregs;
implanting the donor organ into a recipient patient;
prior to, during, or after implantation of the donor organ into the recipient patient, administering the DCregs to the patient, thereby inducing tolerance to the donor organ in the recipient patient.

Clause 17. The method of clause 16, wherein the donor graft tissue is an organ.

Clause 18. The method of clause 17, wherein the organ is selected from: liver, kidney, pancreas, heart, lung, intestine, bone, bone marrow, connective tissue, or a vascularized composite graft.

Clause 19. The method of clause 17, wherein the organ is liver.

Clause 20. The method of clause 17, wherein the organ is lung, pancreas, intestine, heart, or kidney.

Clause 21. The method of any one of clauses 16-20, wherein the donor organ is an allograft.

Clause 22. The method of any one of clauses 16-21, wherein the DCregs are prepared by culturing the monocytes in media containing IL4 and GM-CSF.

Clause 23. The method of clause 22, wherein the media further comprises one or more additional immunosuppressive compounds, such as IL-10, Vitamin D3, a combination of IL10 and Vitamin D3, an anti-inflammatory cytokine, or an immunosuppressive or anti-inflammatory drug, such as dexamethasone or rapamycin.

Clause 24. The method of any one of clauses 17-23, wherein the monocytes are obtained by apheresis of the perfusion solution after it passes through the donor organ.

Clause 25. The method of any one of clauses 17-24, wherein the monocytes are obtained by a method comprising elutriation of cells obtained from the perfusion solution.

Clause 26. The method of any one of clauses 17-24, wherein the monocytes are obtained by elutriation of leukocytes obtained by apheresis.

Clause 27. The method of clause 25 or 26, further comprising, after elutriation, affinity-purification of the monocytes by CD14 affinity or depletion of neutrophils by CD15 and/or CD66b affinity, such as by magnetic bead separation.

Clause 28. The method of any one of clauses 16-27, wherein the monocytes are obtained from a back-table perfusate fraction and/or from a container in which the donor organ is transferred from the donor to recipient patient.

Clause 29. The method of any one of clauses 16-28, comprising administering the DCregs to the patient more than once.

Clause 30. The method of any one of clauses 16-29, further comprising administering an immunosuppressant to the patient prior to, during, or after implantation of the donor organ into the recipient patient.

Clause 31. The method of clause 30, wherein a dosage of the immunosuppressant administered to the patient is lowered or discontinued after administration of the DCregs to the patient.

Clause 32. A method of treating a patient having a transplant-associated autoimmune disease, comprising:
obtaining a donor organ from a donor;
perfusing the donor organ with a perfusion solution;
isolating monocytes (e.g., CD14+ monocytes) from the perfusion solution;
differentiating the monocytes to a DCreg phenotype thereby producing DCregs;
implanting the donor organ into a recipient patient;
prior to, during, or after implantation of the donor organ into the recipient patient, administering the DCregs to the patient, thereby inducing tolerance to the donor organ in the recipient patient.

Clause 33. The method of clause 32, wherein the transplant-associated autoimmune disease is associated with a liver transplant.

### BRIEF DESCRIPTION OF THE DRAWINGS

FIG. 1 provides a flowchart providing method of transplantation of an organ according to the present invention.

FIGS. 2A-2C. Flow cytometry analysis of immature (im) DC/mature (m) DC (pooled on top panels) versus DCreg/DCreg+MPLA (pooled on the bottom panels) (FIGS. 2A and 2B). PDL-1: CD86 MFI ratio of DC cultured from bead-isolated monocytes from Buffy coat (n=7) and deceased donor liver perfusate (n=30) (FIG. 2C). Significances of differences were determined using Tukey's multiple comparisons test. ***p<0.05. Data are mean±SEM.

FIG. 3. Proliferation of allogeneic T cells stimulated with DC populations. DCs were generated from bead-isolated monocytes (n=22). CFSE-labeled allogeneic T cells were co-cultured for 5 days with various populations of DC (1DC: 10T cells). DCs or DCregs were stimulated for 20 h with MPLA to generate mDC and DCregMPLA, respectively. T cell proliferation was measured by CFSE dilution. Tukey's multiple comparisons test. ***p<0.05. Data are mean±SEM.

FIGS. 4A and 4B. Flow cytometry analysis. Cell surface marker expression as Mean Fluorescence Intensity (MFI) by DC populations generated after 7 days of culture from bead-isolated monocytes (n=9) from live donor liver perfusate (LDLP), and percentage of double-positive (CD163/CD141) cells. Data are mean±SEM. imDC, immature DC; DCreg, regulatory DC; mDC, imDC stimulated with MPLA; DCreg+MPLA, DCreg stimulated with MPLA. p<0.05.

FIG. 5. PDL1: CD86 (A) PDL1:CD40 (B) and PDL1:CD86 (C) MFI ratio of DC populations generated after 7 days of culture from bead-isolated monocytes (n=9) from live donor liver perfusate (LDLP). Data are mean±SEM. imDC, immature DC; DCreg, regulatory DC; mDC, imDC stimulated with MPLA; DCreg+MPLA, DCreg stimulated with MPLA. p<0.05.

FIG. 6. Provides graphs showing quantification of multiple cytokines in 7-day cultures revealed comparable cytokine levels in DCreg culture supernatants before and after MPLA stimulation. IL6 (A), TNFα (B), ILp40 (C), and IL10 (D) levels and IL10: TNFα (E) and IL10: IL12p40 (F) ratio in different DC populations supernatants after 7 days of culture from bead-isolated monocytes (n=6) from live donor liver perfusate (LDLP). Data are mean±SEM. imDC, immature DC; DCreg, regulatory DC; mDC, imDC stimulated with MPLA; DCreg+MPLA, DCreg stimulated with MPLA. p<0.05.

FIG. 7. Proliferation of allogeneic T cells stimulated with DC populations. DCs were generated from bead-isolated monocytes (n=9). CTV-labeled allogeneic T cells were co-cultured for 4 days with various populations of DC (1DC: 10T cells). DCs or DCregs were stimulated for 20 h with MPLA to generate mDC and DCregMPLA, respectively. % CD8 T cell proliferation (A), % CD4 T cell proliferation (B) and % CD4+CD25+Foxp3+CD127-cells (Treg) (C). Tukey's multiple comparisons test. ***p<0.05. Data are mean±SEM. See, also, TABLE 1.

FIG. 8: Cellular composition of liver perfusate (n=4) before (A) and after apheresis, using Com.TEC (B); elutriation, using ELUTRA (C); cellular isolation through magnetic beads separation, using AutoMacs. SFL/SSC plot of CD14+ fraction (left panel) and CD3+CD15+ fraction (right panel) (D).

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include but are not limited to embodiments “consisting essentially of” and “consisting of” these stated elements or steps. For definitions provided herein, those definitions refer to word forms, cognates and grammatical variants of those words or phrases. Words or phrases not defined specifically herein will take their ordinary meaning according to a person of ordinary skill in the medical, immunological, or pharmaceutical arts. Compounds, including pharmaceutically-active or immunologically-active compounds, including proteins and antibody reagents, may be listed in their recognized form, such as IL10, but are intended to include functional equivalents thereof as are recognized, such as, for example and without limitation: pharmaceutically-acceptable salts; equivalent amino acid sequence variants and homologs; polyclonal antibodies, monoclonal antibody fragments, scFvs, nanobodies, or other epitope-binding compounds; or prodrugs.

Provided herein is a method of preparing DCregs (regulatory Dendritic Cells) for use in tissue transplantation, as well as a transplantation method. DCregs obtained from the monocyte fraction isolated from fluid obtained from perfusion of the tissue resected from deceased (e.g., brain-dead-heart-beating) or living donors. The donor and recipient may be human. The tissue may be liver tissue, e.g., as described in the examples below. Monocytes may be obtained from the liver perfusate by apheresis, e.g., leukapheresis (e.g., Com.TEC, Fresenius-Kabi), followed by a step of elutriation, ELUTRA, Terumo BCT), or magnetic bead separation (e.g., MACSProdigy, Miltenyi Biotec). DCregs may be obtained by culturing monocytes for about one week with cytokines IL4, GM-CSF, IL10 and Vitamin-D3 according to an established protocol (see, e.g., Macedo C, Tran L M, Zahorchak A F, Dai H, Gu X, Ravichandran R, Mohanakumar T, Elinoff B, Zeevi A, Styn M A, Humar A, Lakkis F G, Metes D M, Thomson A W. Donor-derived regulatory dendritic cells infusion results in host cell cross-dressing and T cell subset changes in prospective living donor liver transplant recipients. Am J Transplant. 2021 July; 21 (7):2372-2386). Alternative methods described in the literature for generation of DCregs could also be employed (e.g., use of dexamethasone, or IL-10 alone) DCregs prepared according to methods described herein may express a tolerogenic gene transcriptional profile, high cell surface programmed death ligand-1 (PD-L1):CD86 ratios, high IL-10/no IL-12 productivity, high IL-10/TNF productivity and poor ability to stimulate allogeneic T cell proliferation. DCregs prepared according to methods described herein may be administered to graft tissue, e.g., solid organ recipients to achieve staged immunosuppression withdrawal and operational tolerance (see, e.g., Thomson A W, Ezzelarab M B. Regulatory dendritic cells: profiling, targeting, and therapeutic application. Curr Opin Organ Transplant. 2018 October; 23 (5):538-545).

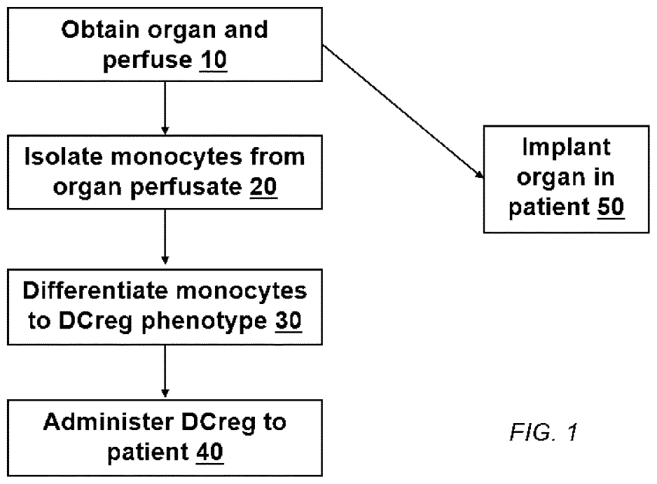

Referring to FIG. 1, according to one aspect of a transplantation method provided herein, an organ may be obtained from a donor and is perfused 10. The donor may be alive or deceased. The tissue may be perfused as a typical part of the organ preservation method. The tissue may be perfused under normothermic (NMP) or subnormothermic (SMP) temperatures (see, e.g., Schlegel A, Muller X, Dutkowski P. Machine perfusion strategies in liver transplantation. Hepatobiliary Surg Nutr. 2019 October; 8 (5):490-501). For the methods described herein any perfusion solution may be employed, such as, for example and without limitation: University of Wisconsin (UW) solution, histidine-tryptophan-ketoglutarate (HTK) solution, or a blood-based perfusate (Organox®, Transmedics®, Liver Assist®). See, e.g., Stewart Z A. UW solution: still the “gold standard” for liver transplantation. Am J Transplant. 2015 February; 15 (2):295-6). The organ may optionally be stored cold.

The perfusate may be obtained in “back-table” procedures, which often are performed in the surgical environment immediately prior to implantation of graft tissue. Such back table procedures may include perfusion of the organ, among other tasks. Thus “back-table perfusate” or a “back-table fraction” of perfusate includes perfusate obtained during such back table procedures prior to, e.g., immediately prior to, implantation in a graft recipient.

Organs often transplanted include, for example and without limitation: liver, kidney, pancreas, heart, lung, intestine, corneas, middle ear, skin, bone, bone marrow, heart valves, connective tissue, vascularized composite allografts (transplant of several structures that may include skin, uterus, bone, muscles, blood vessels, nerves and connective tissue). To the extent that monocytes may be obtained in adequate number from the organ to be transplanted by perfusion (for purposes herein to include washing), the methods described herein may be employed.

Referring to FIG. 1, monocytes may be isolated from perfusate 20. This may be achieved by apheresis, followed by affinity purification (e.g., magnetic bead separation or panning) or elutriation, which is shown below to be more practically applicable for larger-scale preparations techniques. Elutriation is a technique that separates particles based on size and density by sedimenting particles such as cells with concurrent application of fluid flow in the opposite direction of sedimentation (e.g., counterflow elutriation). Centrifugal elutriation is a centrifugation technique performed with use of an elutriation rotor in a centrifuge. Specialized elutriation devices are available to achieve this, such as the ELUTRA instrument (TERUMO) or the JE-5.0 Elutriator Rotor (Beckman Coulter).

Next, the thus-obtained monocytes are differentiated into DCregs 30, e.g., by culture in culture media containing IL4, IL10, GM-CSF, and/or Vitamin D3, e.g., as described in Macedo C, et al. (Am J Transplant. 2021 July; 21 (7):2372-2386). DCregs may then be administered to the allograft recipient 40, typically after implantation of the allograft 50 in the recipient. Traditional immunosuppressants (e.g., anti-thymocyte globulin, tacrolimus, cyclosporine, mycophenolate mofetil, azathioprine, everolimus, sirolimus, and/or glucocorticoids such as methylprednisolone, prednisone, or dexamethasone) may be administered to the patient prior to administration of the DCregs to the patient given it takes about a week to prepare the donor DCregs from the allograft perfusate. After administration of the donor DCregs to the graft recipient patient, the traditional immunosuppressant may eventually be discontinued, continued at the same or lower doses, or weaned in the recipient patient.

Various cell culture additives, e.g., cytokines and immune-regulatory compounds, such as proteins and peptides, are referenced herein, such as IL4 and GM-CSF. Affinity-purifying reagents, such as antibodies, e.g., binding CD15 and/or CD66b are readily-available commercially, optionally pre-conjugated with a suitable bead, e.g., from Miltenyi Biotech.

Cell growth media or medium is a solution used to support growth and optionally expansion of a cell population in vitro. A large variety of media are available commercially. Often serum, such as fetal bovine serum, is used to promote cell growth. Additional factors, including buffers, co-factors, antimicrobials, salts, cellular extracts and/or amino acids, carbon source(s), among other factors, are included in various media. For different mammalian cell types, different ingredients are present in any given medium, which often is optimized to promote growth and expansion of that given cell type. The end-use of the cells also is taken into consideration when formulating and optimizing a particular medium. For example, to produce cells or conditioned medium for use in human patients, “xeno free” (xenogeneic-free) media may be used, which contains no natural products, e.g., proteins or other potentially antigenic substances, obtained from non-human sources, such as fetal bovine serum, or cell extracts. “Xeno-free” relates to the species in which the conditioned media is to be used, and therefore if the conditioned media is to be used in humans or for propagating human cells, the media contains no non-human products (e.g. proteins or serum), while if the conditioned media is to be used for veterinary purposes, e.g. in dogs or for propagating dog cells, the media contains no products from a different species. A non-limiting example of a useful, xeno-free, medium for differentiation of perfused donor monocytes to DCregs is CellGenix GMP Dendritic Cell Medium, Serum-free (CellGenix). Other media for differentiation of perfused monocytes to DCregs are commercially available and/or described in the literature.

As indicated above, despite their clear value in transplant tolerance, obtaining DCs from peripheral blood is a significant rate-limiting barrier to this technology reaching its full clinical promise. First, human donor DCregs previously were only obtainable from donors' PBLs, meaning tissue-specific DCregs were not available. Obtaining DCregs from tissue-specific monocyte populations is a distinct benefit. Second, DCregs were not previously obtained in the common situation where the donor is deceased. Deriving DCregs from monocytes from organ perfusate opens up a broader category of situations where donor DCregs may be used to suppress recipient immune response to allograft tissue. Lastly, methods are provided herein for large-scale processing of perfusate, including an elutriation step that removes the need for expensive magnetic bead separation methods, or similar affinity-based methods.

In sum, provided herein is a method of preparing DCregs from a novel source of monocytes. The method expands the cohort of tissue donors for which a practitioner can use donor DCreg-based immunosuppression techniques to reduce tissue rejection. Current methods involve obtaining precursor monocytes from peripheral blood of a donor. This approach presents the following advantages: the incidence of transplantations from deceased donors is higher than those from living donors. As consequence, the cohort of patients eligible for this treatment would be greatly increased. In the case of liver, on average, liver perfusate contains about 1-5×10⁸ CD14+ cells, representing a rich starting cellular product as for cell numbers. Other organs typically used in transplantation, such as heart, lung, intestine, and kidney, also comprise significant numbers of tissue monocytes that can be washed out from perfusate.

### Examples

In Vitro Generation of Regulatory Dendritic Cells (DCregs) from Deceased and Living Donor Liver Transplant Liver Perfusate (LP): The use of DCregs from liver perfusate, not only increases the number of DCreg-treatable individuals (inducing operational tolerance also in the cohort of patients that receive livers from deceased donors) but also emerged that they have highly potent regulatory properties compared DCregs obtained from peripheral blood. These cells may prove ideal for future cellular therapy in prospective transplant recipients and may facilitate the safe, complete withdrawal of immunosuppression without jeopardizing allograft function or histology. Naturally occurring regulatory immune cells, either innate or adaptive, are rare and critically regulate T cell immunity, promote antigen (Ag)-specific T cell hypo-responsiveness, and prevent adverse immune reactions in the healthy steady-state. In addition to regulatory T cells (Treg) and blood monocyte-derived DCregs, both being evaluated for cell-based therapy in organ transplantation, a compelling rationale has emerged for testing new, modified protocols for ex vivo generation of DCregs from monocyte precursors isolated from living or deceased donor Liver Perfusate (LP), with superior regulatory properties (LP-DCreg).

Collection of Liver Perfusate from Deceased Donor (DDLP): Monocytes were isolated from the product of perfusion of the liver excised from brain-dead-heart-beating donors. All graft procurements were performed at the same standard set of perfusion pressure and temperature, with identical surgical instruments, skilled medical and nursing staff, and the same procedure. During liver procurement, the aorta was clamped, and the liver flushed in situ via the hepatic artery, with up to 6 L of preservation Celsior solution (SangStat Medical Corporation, Fremont, CA, USA) to exsanguinate the deceased donor (Fraction 1 and Fraction 2). LP collection was performed under sterile conditions, via a vacuum-pump suction system directly into an auto-transfusion reservoir (ATR) (Fresenius-Kabi, Bad Homburg, DE) filled with 10% acid citrate dextrose (ACD) (Fresenius-Kabi) and 50 mM of EDTA and using a sterile tubing system connected to a sterile draining system composed of a suction tube with a cannula. At the end of the procurement, reservoirs were closed with appropriate sterile lids and were secured with hose clamps. Reservoirs containing the liver perfusate (Fr1 and Fr2) were transferred to ISMETT in a thermally insulated container to guarantee a constant temperature of about 4 °C. Once in the operating room, the liver was perfused through the portal vein with 1 to 2 L of filled with a solution of 10% ACD-50 mM EDTA. The back-table fraction was kept in the Steri-Drape Isolation Bag until the organ was transplanted. After the liver was removed from the 3 M bag for engraftment, the residual perfusate in the Isolation Bag (Fraction 3) was aspirated into an autotransfusion reservoir (ATR) (Fresenius-Kabi, Bad Homburg, DE), and maintained at 4 °C until processing.

Collection of Liver Perfusate from Living Donor (LDLP): The study is designed for the procurement of waste liver perfusate from adult male and female living donors. The source of LDLT LP is obtained from adult living donors that meet the study inclusion and exclusion criteria. The liver was perfused with 1 to 2 L of filled with a solution of 10% ACD-50 mM EDTA. The back-table fraction was kept in the Steri-Drape Isolation Bag until the organ was transplanted. After the liver was removed from the 3 M bag for engraftment, the residual perfusate in the Isolation Bag was transferred to a reservoir and maintained at 4 °C until processing.

Isolation of Monocytes from LP Monocyte (CD14+) Precursors and DCreg Generation

Isolation of monocytes from DDLP (small-scale):37 samples were processed, of which n=7 healthy donors' peripheral blood buffy-coat, and n=30 deceased donors' liver perfusates. It was demonstrated that a method starting from living donors' apheresis product is applicable to CD14+ monocytes isolated from deceased donors' liver perfusates. DCregs were obtained with comparable regulatory phenotype and function to DCregs from apheresis products. Briefly, monocytes were isolated from each liver perfusate. For small-scale studies, we used the back-table fraction (Fraction 3), which is the last fraction perfused from the resected liver just before engraftment. After concentration by centrifugation (2000 rpm, 10 minutes, 10 °C) and Ficol gradient, CD14+ monocytes were isolated with magnetic immune beads and the cell separation unit AutoMACS Cell Separation (Miltenyi Biotec, Germany). Subsequently, CD14+ monocytes were cultured for 7 days in DC media, generating immature DCs (imDCs) and DCregs. As a comparison, we used CD14+ monocytes derived from the buffy coat obtained from the peripheral blood of healthy donors.

Briefly, to generate DCregs, the monocytes were suspended at 106/mL in GMP DC medium (CellGenix, Freiburg, Germany) containing interleukin (IL)-4 (1000 IU/mL CellGenix), granulocyte macrophage-colony stimulating factor (GM-CSF) (1000 IU/mL; Partner Therapeutics, Lynnwood, WA, USA) and Vitamin D3 (20 nmol/L, Millipore Sigma, St. Louis, MO, USA) and incubated in 5% CO₂ at 37 °C. On day 4, DC culture medium containing IL-4, GM-CSF and IL-10 (60 ng/ML; Peptrotech, Rocky Hill, NJ, USA) was added to each well and on day 6, DC culture medium containing IL-4 and GM-CSF was added to each flask (see, e.g., Zahorchak A F, Macedo C, Hamm D E, Butterfield L H, Metes D M, Thomson A W. High PD-L1/CD86 MFI ratio and IL-10 secretion characterize human regulatory dendritic cells generated for clinical testing in organ transplantation. Cell Immunol. 2018 January; 323:9-18 and Zahorchak A F, DeRiggi M L, Muzzio J L, Sutherland V, Humar A, Lakkis F G, Hsu Y S, Thomson A W. Manufacturing and validation of Good Manufacturing Practice-compliant regulatory dendritic cells for infusion into organ transplant recipients. Cytotherapy. 2023 April; 25 (4):432-441).

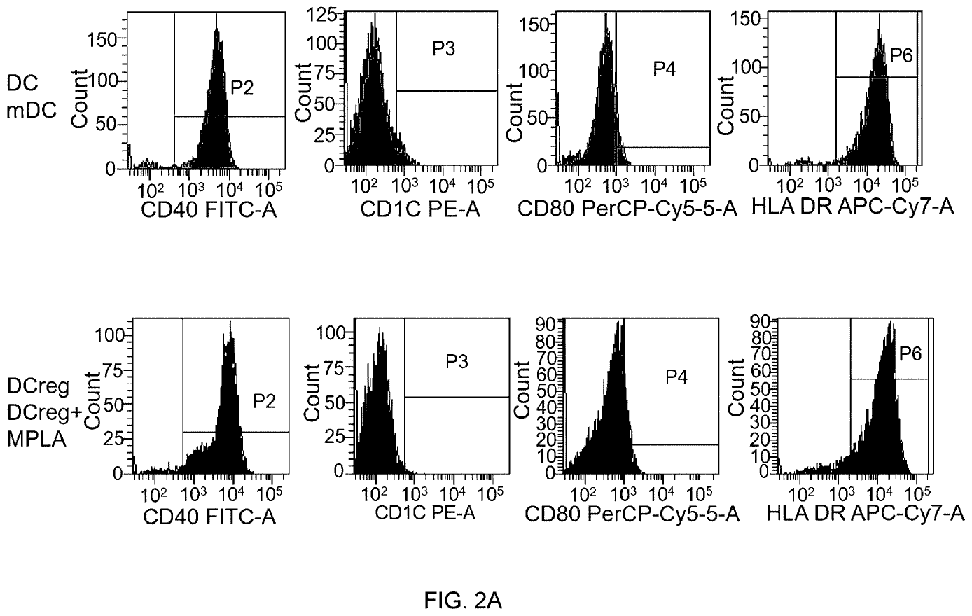

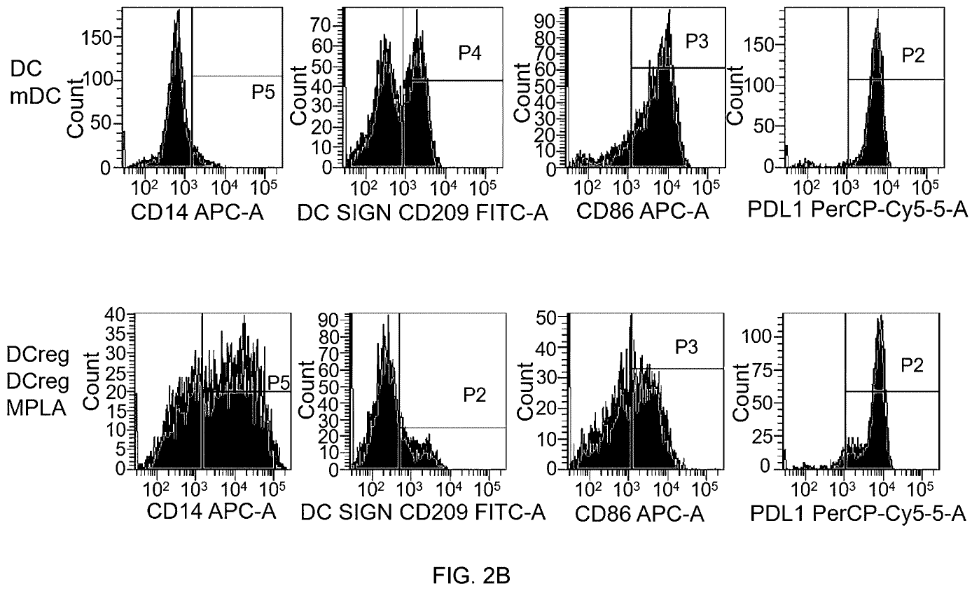

On day 6, a potent immunostimulant (Toll-like receptor 4 ligand) of bacterial origin, MPLA was added. Upon differentiation, cells were harvested and analyzed for phenotypic and functional assays. The cell surface phenotype of DC populations was analyzed by Flow cytometry analysis. We addressed the expression of common markers of maturation such as CD40, CD1c, CD80, HLA-DR, CD14, DC-SIGN, CD86, and PD-L1. As shown in FIG. 2A, markers like CD40, CD1c, CD80, and HLA-DR were not differentially expressed between DCs or DCregs. By contrast, CD14 and DC-SIGN were downregulated by mDC but not by DCregs, as expected as CD14 is downregulated by monocytes-derived DCs. As expected, we noted that CD86 was only mildly represented by DCregs, whilst was nicely expressed by mDC. Likewise, DCregs were characterized by a higher Mean Fluorescence Intensity (MFI) value for the co-inhibitory ligand PD-L1 than mDCs.

One of the release criteria for the clinical use of DCregs is the level of expression of PD-L1 compared to CD86 with a cutoff ratio of 2 or above. It was showed that DCregs expressed comparatively low levels of CD86 and higher PD-L1 than DCs. Monocytes from buffy coat were used as a comparison. In FIG. 2A, it is shown that qualitatively, DCregs from liver perfusate (n=30) equal buffy coat (n=7) derived DCregs, and the PD-L1/CD86 MFI ratio for DCregs (+/−MPLA) is always above the cutoff of 2 (FIG. 2B).

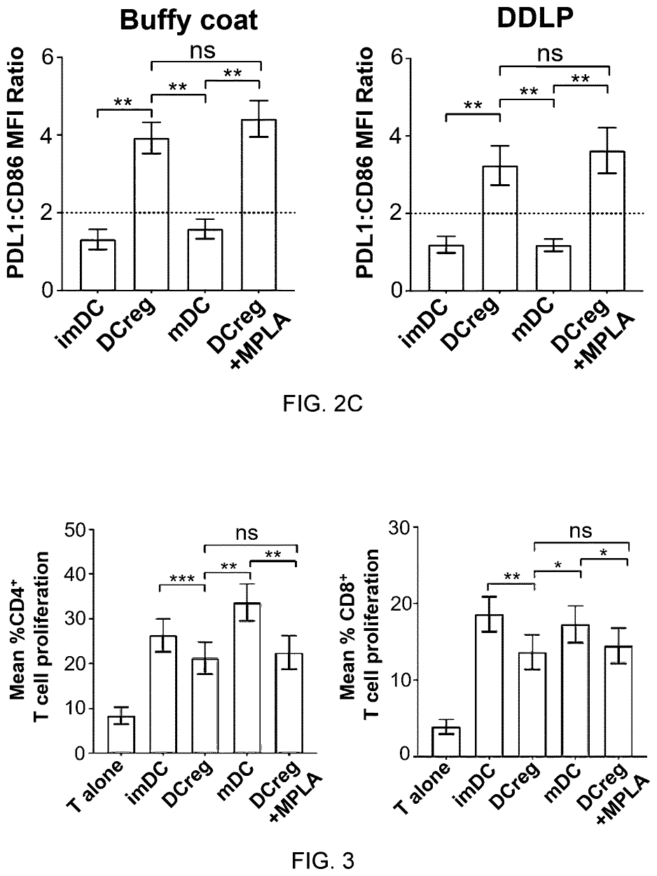

Functional assays were performed to evaluate if the DCreg generated were weak stimulators of allogeneic T cell proliferation. To evaluate T cell proliferation, allogeneic T cells labeled with carboxyfluorescein succinimidyl ester (CFSE; 2 μM; Invitrogen, Waltham, MA) were co-cultured for 5 days at 37 °C in 5% CO₂ in the air with DC (1DC: 10T cell ratio). As shown in FIG. 3, DCregs induced significantly lower levels of CD4+ and CD8+ T cell proliferation than mDC. Further, the stimulation with MPLA didn't change their ability to reduce T cell proliferation compared to control MPLA-stimulated DC. These results characterize DCregs as weak allostimulatory antigen-presenting cells and further demonstrate their ability to resist functional maturation. Finally, these results were compared with DC generated by monocytes isolated from human leukapheresis buffy coat PBMCs, confirming that the cellular component obtained from liver perfusate showed comparable properties to leukapheresis products (data not shown).

To conclude, it is demonstrated that CD14+ monocytes obtained from the liver perfusate of deceased (or live) donors can be used as a starting cellular input for the generation of DCregs with phenotypic and functional characteristics compliant with the DCregs described by Thomson et al. (Macedo C, Tran L M, Zahorchak A F, Dai H, Gu X, Ravichandran R, Mohanakumar T, Elinoff B, Zeevi A, Styn M A, Humar A, Lakkis F G, Metes D M, Thomson A W. Donor-derived regulatory dendritic cell infusion results in host cell cross-dressing and T cell subset changes in prospective living donor liver transplant recipients. Am J Transplant. 2021 July; 21 (7):2372-2386)

Illustrative small-scale and large-scale methods are provided for isolation of monocytes.

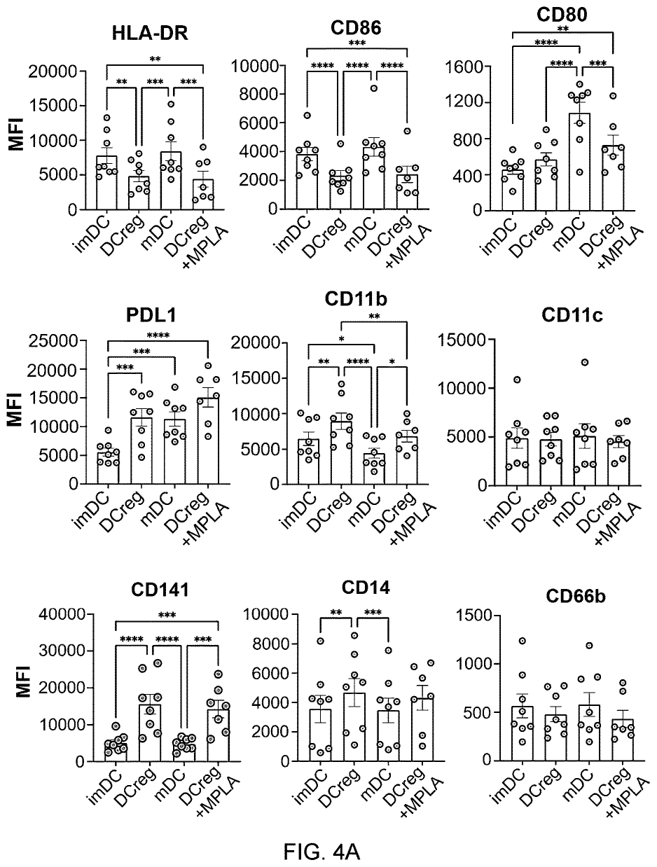

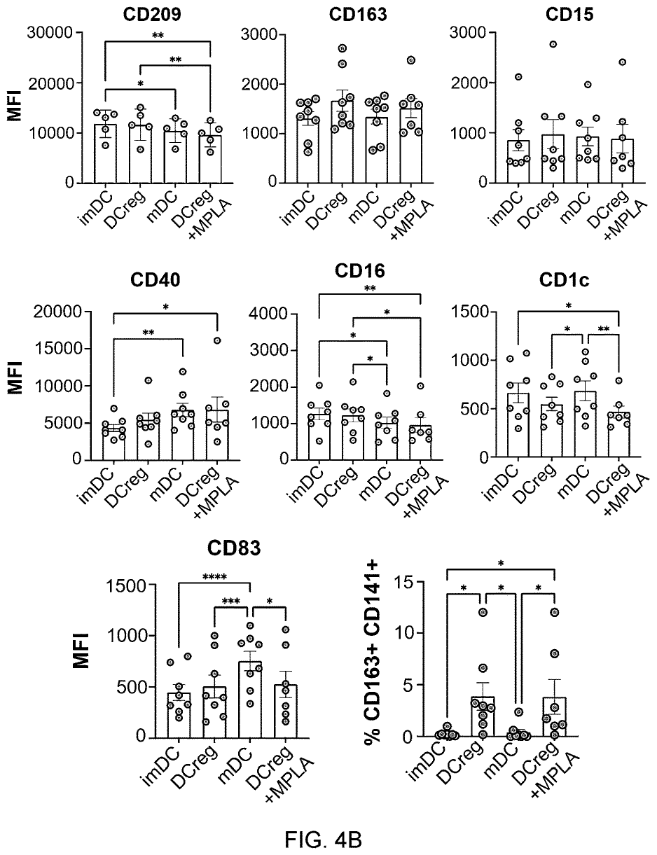

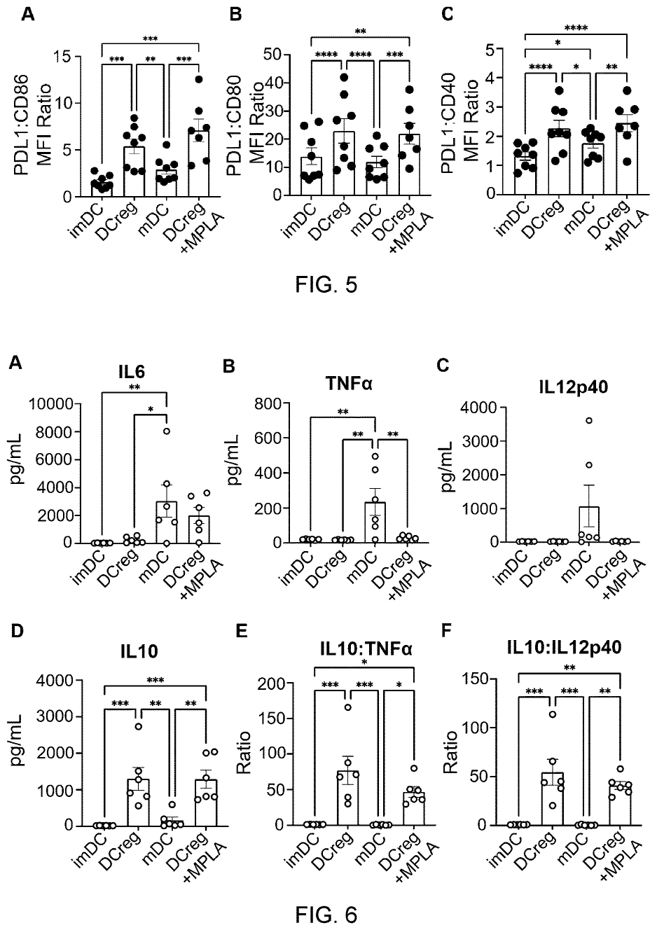

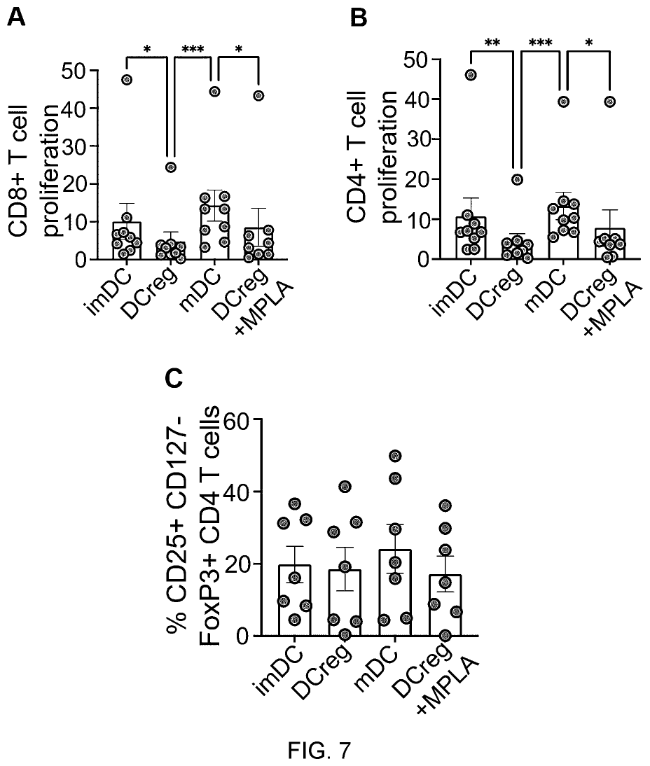

Isolation of monocytes from LDLP (small-scale): Nine additional liver perfusates were processed from living donors, using the described isolation protocol. LP-DCregs showed >80% viability, >90% purity (lin-HLA-DR+CD11c+) with <1% T or B lymphocyte contamination. Recovery rates were higher for LP-DCreg (28-31%) than DCreg generated from blood monocytes (20-23%). LP-DCreg (CD141+CD163+) were phenotypically immature and resisted maturation when exposed to a potent pro-inflammatory stimulus (the Toll-like receptor 4 ligand monophosphoryl lipid A; MPLA), maintaining expression of HLA-DR, CD11b, CD11c, CD40, CD86 and programmed death ligand-1 (PD-L1)>90% (FIGS. 4A and 4B). However, co-inhibitory: co-stimulatory molecule MFI expression ratios, PD-L1: CD86, PD-L1: CD80, and PD-L1: CD40, were all elevated significantly on LP-DCreg compared to immature DC (FIG. 5). Quantification of multiple cytokines in 7-day cultures revealed comparable cytokine levels in DCreg culture supernatants before and after MPLA stimulation. We found minimal levels of TNFα and IL12p40 and high levels of IL10 in DCreg and DCreg+MPLA compared to immature DC and mature DC cultures resulting in high IL10: TNFα and high IL10: IL12p40 ratios (FIG. 6). Functional assessment performed in MLR assay revealed that LP-DCregs induced T cell unresponsiveness by suppression of effector CD4 and CD8 T cell proliferation and induction of Tregs (CD4+CD25+Foxp3+CD127-), even after TLR4 ligation with MPLA (FIG. 7, Table 1).

TABLE 1

| matDC:CTV Pan T (1:5) | % CD4PROL | % CD8PROL |
|---|---|---|
| No DCreg, no block Ab | 16 | 21.6 |
| No DCreg + PDL1, PD1 | 23.6 | 30 |
| No DCreg + IL10R | 16.7 | 23.2 |
| No DCreg + PDL1, PD1, IL10R | 20 | 26.3 |
| No DCreg + IgG | 17.7 | 24.4 |
| matDC/DCreg (1:1) no block Ab | 5.53 | 9.87 |
| matDC/DCreg (1:1) + PDL1, PD1 | 10.5 | 16.3 |
| matDC/DCreg (1:1) + IL10R | 3.27 | 6.97 |
| matDC/DCreg (1:1) + PDL1, PD1, | 10.1 | 16 |
| IL10R | | |
| matDC/DCreg (1:1) + IgG | 4.78 | 8.64 |
| matDC/DCreg (1:0.5) no block Ab | 21 | 26.7 |
| matDC/DCreg (1:0.5) + PDL1, PD1 | 33.5 | 38.9 |
| matDC/DCreg (1:0.5) + IL10R | 19.6 | 25.6 |
| matDC/DCreg (1:0.5) + PDL1, PD1, | 21.7 | 26 |
| IL10R | | |
| matDC/DCreg (1:0.5) + IgG | 23 | 28.2 |
| matDC/DCreg (1:0.25) no block Ab | 16.2 | 25.6 |
| matDC/DCreg (1:0.25) + PDL1, PD1 | 24.4 | 27.2 |
| matDC/DCreg (1:0.25) + IL10R | 18.4 | 21.7 |
| matDC/DCreg (1:0.25) + PDL1, PD1, | 31.9 | 29.8 |
| IL10R | | |
| matDC/DCreg (1:0.25) + IgG | 21.5 | 26.5 |

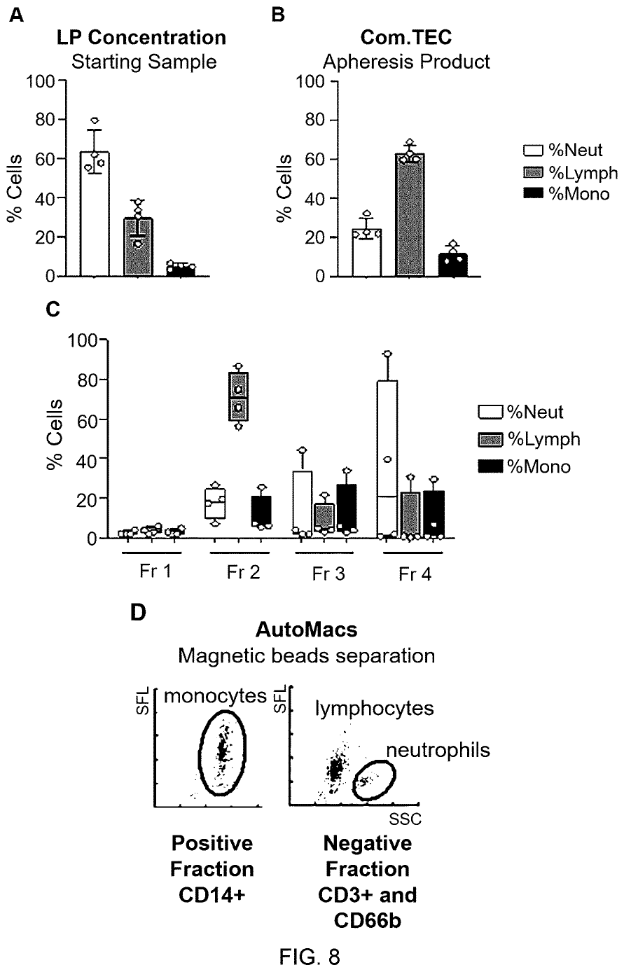

Isolation of monocytes from DDLP (Large-scale): The product of liver perfusion contains billions of viable cells, including monocytes, and consists of a large volume of diluted blood (5-9 liters). LPs were collected in Autotransfusion reservoirs in the presence of anticoagulant ACD (ATR 40, Fresenius-Kabi). The products were first concentrated by centrifugation to a volume of 0.5-1 Liter and on average contain Neutrophils (45-90%), Lymphocytes (20-45%), and Monocytes (5-10%) (FIG. 8(A)). The three factions were pooled together and were checked through Coulter Counter (hematology analyzers). The HCT was brought to a range value comprised between 35% to 48% adding a solution of 10% ACD-50 mM EDTA. Subsequently, the sample was transferred under a laminar hood into a 2×2Lt cell collection bag (Fresenius-Kabi Cat #9007341 BMSC-BAG BONE) and processed using the circuit for leukocyte apheresis C4Y (Fresenius-Kabi Cat #9400301) following the manufacturer's instructions. The LP was processed for at least 3 cycles. At the end of each cycle, one buffy coat fraction was harvested and analyzed through Coulter Counter to check their composition. Usually starting from the 4^{th} cycle, the percentage of contaminant neutrophils in the buffy coat is too high and therefore discarded. The buffy coat fractions with high-purity lymphomonocytes and low contamination of granulocytes were pooled. As shown in FIG. 8(B), Com.TEC apheresis instruments produced a higher enrichment of monocytes and a reduction of the percentage of lymphocytes and neutrophils, but it still contained >15% of neutrophils. Although reducing the contamination of this population was successful, as aforementioned the percentage of neutrophils remains too high to use elutriation to obtain a monocyte pure population. In the presence of >3% of granulocytes, the process of elutriation failed, and monocytes could not be separated in any fraction (FIG. 8(C)). Direct purification of monocytes using (e.g.) CD15 and/or CD66b beads (neutrophil markers) conjugated to magnetic beads was used to further purify the CD14+ monocytes. Flow cytometry or other affinity purification methods also may be used to separate cell populations. Cells were isolated using the large-scale research-grade instrument AutoMACS (Miltenyi Biotec) obtaining a >90% pure population of monocytes (FIG. 8(D)). The median number of purified monocytes that we obtained was 1-5×10⁸, a remarkable quantity for clinical downstream applications. It is expected that the elutriation process can be optimized to remove the need for DC15/CD66b depletion.

The present invention has been described with reference to certain exemplary embodiments. However, it will be recognized by those of ordinary skill in the art that various substitutions, modifications, or combinations of any of the exemplary embodiments may be made without departing from the spirit and scope of the invention. Thus, the invention is not limited by the description of the exemplary embodiments, but rather by the appended claims as originally filed.

1. A method of preparing donor regulatory dendritic cells (DCregs), comprising, isolating monocytes from perfusate obtained from perfusion of donor tissue, and differentiating the monocytes to a DCreg phenotype.

2. The method of claim 1, wherein the donor graft tissue is an organ.

3. The method of claim 2, wherein the organ is selected from: liver, kidney, pancreas, heart, lung, intestine, bone, bone marrow, connective tissue, or a vascularized composite graft.

4-5. (canceled)

6. The method of claim 1, wherein the donor organ is an allograft.

7. The method of claim 1, wherein the DCregs are prepared by culturing the monocytes in media containing IL4 and GM-CSF.

8. The method of claim 7, wherein the media further comprises one or more additional immunosuppressive compounds selected from IL-10, Vitamin D3, a combination of IL10 and Vitamin D3, an anti-inflammatory cytokine, or an immunosuppressive or anti-inflammatory drug.

9. The method of claim 1, wherein the monocytes are obtained by apheresis of the perfusion solution after it passes through the donor organ.

10. The method of claim 1, wherein the monocytes are obtained by a method comprising;
elutriation of cells obtained from the perfusion solution; or
elutriation of leukocytes obtained by apheresis.

11. (canceled)

12. The method of claim 10, further comprising, after elutriation, affinity-purification of the monocytes by CD14 affinity or depletion of neutrophils by CD15 and/or CD66b affinity.

13. The method of any one-s wherein the monocytes are obtained from a back-table perfusate fraction and/or from a container in which the donor organ is transferred from the donor to recipient patient.

14-15. (canceled)

16. A transplantation method comprising:
obtaining a donor organ from a donor;
perfusing the donor organ with a perfusion solution;
isolating monocytes from the perfusion solution;
differentiating the monocytes to a DCreg phenotype thereby producing DCregs;
implanting the donor organ into a recipient patient;
prior to, during, or after implantation of the donor organ into the recipient patient, administering the DCregs to the patient, thereby inducing tolerance to the donor organ in the recipient patient.

17. The method of claim 16, wherein the donor graft tissue is an organ selected from: liver, kidney, pancreas, heart, lung, intestine, bone, bone marrow, connective tissue, or a vascularized composite graft.

18-20. (canceled)

21. The method of claim 16, wherein the donor organ is an allograft.

22-27. (canceled)

28. The method of claim 16, wherein the monocytes are obtained from a back-table perfusate fraction and/or from a container in which the donor organ is transferred from the donor to recipient patient.

29. The method of claim 16, comprising administering the DCregs to the patient more than once.

30. The method of claim 16, further comprising administering an immunosuppressant to the patient prior to, during, or after implantation of the donor organ into the recipient patient.

31. The method of claim 30, wherein a dosage of the immunosuppressant administered to the patient is lowered or discontinued after administration of the DCregs to the patient.

32. A method of treating a patient having a transplant-associated autoimmune disease, comprising:
obtaining a donor organ from a donor;
perfusing the donor organ with a perfusion solution;
isolating monocytes from the perfusion solution;
differentiating the monocytes to a DCreg phenotype thereby producing DCregs;
implanting the donor organ into a recipient patient;
prior to, during, or after implantation of the donor organ into the recipient patient, administering the DCregs to the patient, thereby inducing tolerance to the donor organ in the recipient patient.

33. The method of claim 32, wherein the transplant-associated autoimmune disease is associated with a liver transplant.

34. The method of claim 8, wherein the immunosuppressive or anti-inflammatory drug is dexamethasone or rapamycin.
